(19) United States
(12) Patent Application Publication
(10) Pub. No.: US 2004/0110828 A1
(43) Pub. Date: Jun. 10, 2004

(54) Tetrahydrocannabinol compositions and methods of manufacture and use thereof

(76) Inventors: Dipak K. Chowdhury, Lexington, KY (US); Prasad Challapalli, Lexington, KY (US); B. Ram Murty, Lexington, KY (US)

Correspondence Address: Dr. Dipak K. Chowdbury, Murty Pharmaceuticals, Inc., 518 Codell Drive, Lexington, KY, 40509, US

(21) Appl. No.: 10/724,337

(22) Filed: Nov. 28, 2003

Related U.S. Application Data
(60) Provisional application No. 60/429,672, filed on Nov. 27, 2002.

(51) Int. Cl. A61K 31/353

(52) U.S. Cl. 514/454

(57) ABSTRACT

Disclosed is an injectable pharmaceutical formulation containing tetrahydrocannabinol and certain excipients. Also disclosed is how to make and use the formulation.

### PRIORITY

Priority is claimed on the basis of provisional application No. 60/429,672, filed Nov. 27, 2002, which is fully incorporated herein by reference in its entirety.

### STATEMENT REGARDING FEDERAL SPONSORSHIP

Not applicable

### FIELD OF THE INVENTION

The invention relates to tetrahydrocannabinol compositions and methods of manufacture and use thereof.

### BACKGROUND OF THE INVENTION

Hundreds of medically useful compounds are discovered each year, but clinical use of these drugs is possible only if a drug delivery vehicle is developed to transport them to their therapeutic target in the human body. This problem is particularly critical for water-insoluble or poorly soluble drugs. For such hydrophobic compounds, direct injection may be highly dangerous and can result in hemolysis, phlebitis, hypersensitivity, organ failure, or death. Tetrahydrocannabinol (“THC”) is one such compound.

While THC, especially Delta 9-tetrahydrocannabinol, is useful in treating, lessening, or ameliorating emesis, anorexia, or chronic or AIDS-related wasting syndrome in a subject in which it is desired to treat, to lessen, or to ameliorate emesis, anorexia, or chronic or AIDS-related wasting syndrome, THC is so poorly soluble in water that it is difficult to prepare therapeutically useful aqueous formulations of THC at THC concentrations such as 2 micrograms per milliliter. It is an object of the invention to provide a therapeutically useful aqueous formulation of THC.

THC is effective in treating pain, nausea and vomiting associated with chemotherapy and severe weight loss associated with AIDS. It has been recommended that THC be administered to patients who have not responded to other therapies for these conditions.

There is a dearth of THC-based pharmaceuticals on the market. One marketed THC-based pharmaceutical is available in capsule dosage form for oral administration and was approved by the US Food and Drug Administration for indications including emesis associated with chemotherapy and severe weight loss associated with AIDS. However, oral therapy frequently results in a poor or partial response. This may be due to the limited aqueous solubility of THC and its extensive first-pass metabolism following oral administration. Thus, absolute bioavailability of Delta 9-THC is low. In addition, fasting or food deprivation can decrease the rate of absorption of THC from the currently marketed sesame oil capsules. There is also large inter-subject variability in absorption. For this reason it may be important to titrate the THC dose on an individual basis, since the drug has biphasic activity and a narrow therapeutic index.

THC has been utilized throughout the world for centuries. THC appears to be efficacious for the amelioration of nausea due to chemotherapy and for the management of chronic pain. THC can even be utilized to reduce the devastating inflammatory process caused by acute injury to the brain or spinal cord.

Physiologically active constituents of marijuana include the two tetrahydrcannabinols, Delta 9-tetrahydrocannabinol and Delta 8-tetrahydrocannabinol. Water-soluble derivatives have been obtained by esterification of the phenolic group.

The pharmacokinetics of THC varies with the route of administration. When smoked, Delta 9-THC is rapidly absorbed by the blood in the lungs. Oral absorption of THC is less rapid than from the lungs. The disappearance of Delta 9-THC from the blood following intravenous (IV) administration is biphasic. High blood levels fall rapidly for the first 30 minutes as the Delta 9-THC distributes to tissues with high blood flow. After the initial high distribution, the blood level falls much more slowly with a half-life of 19 hours or more. After an IV injection of a single dose of Delta 9-THC, approximately 25-30 percent of the compound and its metabolites remain in the body for one week. In addition, blood levels of Delta 9-THC are higher and last longer when given in an oily solution than in an ethyl alcohol solution. This suggests that cannabis taken with food mixtures containing fat is better absorbed.

An important difference between smoking and ingestion as means of THC administration is that when cannabinoids are absorbed from the gut, the blood containing them first goes directly through the liver. The liver rapidly clears the Delta 9-THC from the blood and enzymatically changes much of the Delta 9-THC to other metabolites before much of the Delta 9-THC can reach the brain. A large proportion is metabolized to 11-hydroxy delta 9-THC. When taken orally, two to three times more Delta 9-THC is required to obtain equivalent acute psychological and physiological effects, as compared with THC administered by smoking.

Apart from this, patients who suffer from severe pain after surgery are given painkillers, such as morphine, which are known to induce vomiting. To reduce vomiting, it is essential to administer an antiemetic agent that can act rapidly. In an attempt to overcome such problems, transdermal patches have been proposed. For example, U.S. Pat. No. 6,113,940 discloses a patch-like device by means of which cannabinoids are delivered transdermally. It can be seen, however, that transdermal approaches have certain limitations, such as variation in the amount of THC released. Since THC has a narrow therapeutic index, it may reach toxic levels if there is too much variation of release.

It is therefore an object of the invention to provide a composition useful for safe, reliable and effective delivery of THC.

References concerning the foregoing background include the following:

Physician's Desk Reference. 50th ed, Medical economics data, Oradell, N. J., 611 (1966).

Cohen, S. In Marijuana Research Findings, Petersen, R. C. (Ed), NIDA RES. Monogr. 31, DHHS, 1980.

Scadler, B. M., Wall, M. E., Perez-Reyes. In the Cannabinoids: Chemical, Pharmacologic and therapeutics aspects, Agurell, S., Dewey, W. L., Willette, R. E. (Eds). Academic New York, pp227, 1984 4. Joy, J. E., Watson, S. J. And Benson, J. A. Marijuana and Medicine: Assessing the Science Base. National Academy Press, Washington. D.C. 1999.

Pryor, G. T., and Mitoma, C. Influence of fasting on the absorption and effects of Delta 9-tetrahydrocannabinol after oral administration in sesame oil. Pharmacol. Biochem. Behav. 6: 331-441(1997).

Ohlsson, A., Lindergren J. E., Wahlen, A., Plasma delta 9-tetrahydrocannabinol concentrations and clinical effects after oral and intravenous administration and smoking. Clin. Pharmacol. Ther. 28: 409-416(1980).

The Merck Index, Merck and co. Inc. Rahway, New Jersy (1989).

Hunt, A. and Jones, R. T. Tolerance and disposition of tetrahydrocannabinol in man Pharmacol. Exp. Ther. 215, 35-44 (1980).

Perez-Reyes, M., Lipton M. A., Timmons M. C. Pharmacology of orally administered Delta 9-THC. Clin. Pharmacol. Ther. 14, 48-55, 1973.

### DESCRIPTION OF THE INVENTION

Accordingly, the invention provides an injectable pharmaceutical composition comprising tetrahydrocannabinol, ethanol, water, and a pharmaceutically acceptable amphiphilic excipient.

The invention provides an injectable pharmaceutical composition comprising tetrahydrocannabinol, ethanol, water, and a pharmaceutically acceptable amphiphilic excipient composition and further comprising a pharmaceutically acceptable excipient salt.

The invention provides an injectable pharmaceutical composition comprising tetrahydrocannabinol, ethanol, water, and a pharmaceutically acceptable amphiphilic excipient composition and further comprising a pharmaceutically acceptable excipient oil.

The invention provides an injectable pharmaceutical composition comprising tetrahydrocannabinol, ethanol, water, and a pharmaceutically acceptable amphiphilic excipient composition and further comprising a pharmaceutically acceptable excipient antioxidant.

The invention provides an injectable pharmaceutical composition comprising tetrahydrocannabinol, ethanol, water, and a pharmaceutically acceptable amphiphilic excipient, wherein the concentration of tetrahydrocannabinol is, by mass, not greater than about 0.35%.

The invention provides an injectable pharmaceutical composition comprising tetrahydrocannabinol, ethanol, water, and a pharmaceutically acceptable amphiphilic excipient, wherein the concentration of ethanol is, by mass, not greater than about 15%.

The invention provides an injectable pharmaceutical composition comprising tetrahydrocannabinol, ethanol, water, and a pharmaceutically acceptable amphiphilic excipient, wherein the concentration of water is, by mass, not greater than about 90%.

The invention provides an injectable pharmaceutical composition comprising tetrahydrocannabinol, ethanol, water, and a pharmaceutically acceptable amphiphilic excipient, wherein the amphiphilic excipient comprises at least one member of the group consisting of: Cremophor EL, Polysorbate 80, Poloxamer 407, Poloxamer 237, PEG 400, Pharmasolve, propylene glycol, and hydroxypropyl beta-cyclodextrin.

The invention provides an injectable pharmaceutical composition comprising tetrahydrocannabinol, ethanol, water, and a pharmaceutically acceptable amphiphilic excipient composition and further comprising a pharmaceutically acceptable excipient salt, wherein the salt comprises sodium chloride or sodium hydroxide.

The invention provides an injectable pharmaceutical composition comprising tetrahydrocannabinol, ethanol, water, and a pharmaceutically acceptable amphiphilic excipient composition and further comprising a pharmaceutically acceptable excipient oil, wherein the oil comprises corn oil.

The invention provides an injectable pharmaceutical composition comprising tetrahydrocannabinol, ethanol, water, and a pharmaceutically acceptable amphiphilic excipient composition and further comprising a pharmaceutically acceptable excipient antioxidant, wherein the antioxidant comprises sodium metabisulfite or ascorbyl palmitate.

The invention provides an injectable pharmaceutical composition comprising tetrahydrocannabinol, ethanol, water, and a pharmaceutically acceptable amphiphilic excipient, wherein the amphiphilic excipient comprises at least one member of the group consisting of Cremophor EL, Polysorbate 80, Poloxamer 407, Poloxamer 237, PEG 400, Pharmasolve, propylene glycol, and hydroxypropyl beta-cyclodextrin; and wherein at least one member of the following group of limitations on concentration obtains: the concentration of Cremophor EL is, by mass, not greater than about 20%; the concentration of Polysorbate 80 is, by mass, not greater than about 15%; the concentration of Poloxamer 407 is, by mass, not greater than about 2.5%; the concentration of Poloxamer 237 is, by mass, not greater than about 5%; the concentration of PEG-400 is, by mass, not greater than about 20%; the concentration of Pharmasolve is, by volume, not greater than about 10%; the concentration of propylene glycol is, by mass, not greater than about 60%; the concentration of hyroxypropyl beta-cyclodextrin is, by mass, not greater than about 30%.

The invention provides an injectable pharmaceutical composition comprising tetrahydrocannabinol, ethanol, water, and a pharmaceutically acceptable amphiphilic excipient composition and further comprising a pharmaceutically acceptable excipient salt, wherein the salt comprises sodium chloride or sodium hydroxide, and wherein the concentration of the salt renders the composition essentially isotonic.

The invention provides an injectable pharmaceutical composition comprising tetrahydrocannabinol, ethanol, water, and a pharmaceutically acceptable amphiphilic excipient composition and further comprising a pharmaceutically acceptable excipient salt, wherein the salt comprises sodium chloride or sodium hydroxide, and wherein the concentration of sodium chloride is, by mass, about 0.9%.

The invention provides an injectable pharmaceutical composition comprising tetrahydrocannabinol, ethanol, water, and a pharmaceutically acceptable amphiphilic excipient composition and further comprising a pharmaceutically acceptable excipient oil, wherein the oil comprises corn oil, and wherein the concentration of corn oil is, by mass, not greater than about 10%.

The invention provides a method for manufacture of an injectable pharmaceutical composition comprising tetrahydrocannabinol, ethanol, water, and a pharmaceutically acceptable amphiphilic excipient, said method comprising the steps of: admixing tetrahydrocannabinol with ethanol to form a first mixture; admixing water with a pharmaceutically acceptable amphiphilic excipient to form a second mixture; and admixing the first mixture with the second mixture to form a third mixture, wherein said third mixture comprises an intermediate or a finished product in the manufacture of the injectable pharmaceutical composition.

The invention provides a method of treating, lessening, or ameliorating emesis, anorexia, or chronic or AIDS-related wasting syndrome in a subject in which it is desired to treat, to lessen, or to ameliorate emesis, anorexia, or chronic or AIDS-related wasting syndrome, said method comprising administering to the subject a therapeutically effective amount of an injectable pharmaceutical composition comprising tetrahydrocannabinol, ethanol, water, and a pharmaceutically acceptable amphiphilic excipient.

When used in connection with the invention, the term “pharmaceutically acceptable” has the meaning customarily accorded it in the pharmaceutical arts. For example, an excipient for which there is a monograph in Handbook of Pharmaceutical Excipients, 4^{th} Edition, published in 2003 by the Pharmaceutical Press and the American Pharmaceutical Association and fully incorporated herein by reference in its entirety, or in any subsequent edition thereof, is a pharmaceutically acceptable excipient.

Hence, an amphiphilic excipient for which there is a monograph in Handbook of Pharmaceutical Excipients, 4^{th} Edition, or in any subsequent edition thereof, is a pharmaceutically acceptable amphiphilic excipient. Likewise, a salt for which there is a monograph in Handbook of Pharmaceutical Excipients, 4^{th} Edition, or in any subsequent edition thereof, is a pharmaceutically acceptable excipient salt. Also, an oil for which there is a monograph in Handbook of Pharmaceutical Excipients, 4^{th} Edition, or in any subsequent edition thereof, is a pharmaceutically acceptable excipient oil. Moreover, an antioxidant for which there is a monograph in Handbook of Pharmaceutical Excipients, 4^{th} Edition, or in any subsequent edition thereof, is a pharmaceutically acceptable excipient antioxidant.

### EXAMPLE “1”

Admixed were the following: THC 0.01 g; Cremophor EL 1.009 g; Polysorbate 80 0.200 g; Water for Injection 7.86 g; Ethanol 0.8 g; Sodium chloride 0.09 g; Ascorbyl palmitate 0.015 g; NaOH to bring final pH to 7. The composition resulting from the admixture of the foregoing was useful as an injectable pharmaceutical composition.

### EXAMPLE “2”

Admixed were the following: THC 0.017 g; Polysorbate 80 0.515 g; Water for Injection 8.74 g; Ethanol 0.417 g; Sodium chloride 0.09 g; Ascorbyl palmitate 0.004 g; PEG 400 0.207 g; NaOH to bring final pH to 7. The composition resulting from the admixture of the foregoing was useful as an injectable pharmaceutical composition.

### EXAMPLE “3”

Admixed were the following: THC 0.0304 g; Polysorbate 80 0.2 g; Water for Injection 0.09 g; Ethanol 2.74 g; Propylene glycol 12.28 g. The composition resulting from the admixture of the foregoing was useful as an injectable pharmaceutical composition.

### EXAMPLE “4”

Admixed were the following: THC 0.0168 g; Water for Injection 7.03 g; Ethanol 0.4 g; Sodium chloride 0.09 g; Poloxamer 407 (7.5%) 3.0 g; Sodium metabisulfite 0.02 g. The composition resulting from the admixture of the foregoing was useful as an injectable pharmaceutical composition.

### EXAMPLE “5”

Admixed were the following: THC 0.012 g; Polysorbate 80 0.2 g; Water for Injection 7.03 g; Ethanol 0.409 g; Sodium chloride 0.09 g; Sodium metabisulfite 0.02 g; Pharmasolve 2.02 g; NaOH to bring final pH to 7.3. The composition resulting from the admixture of the foregoing was useful as an injectable pharmaceutical composition.

### EXAMPLE “6”

Admixed were the following: THC 0.02 g; Water for Injection 8.87 g; Ethanol 0.4 g; Sodium chloride 0.09 g; Sodium metabisulfite 0.02 g; Poloxamer 237 0.5 g; NaOH to bring final pH to 7.1. The composition resulting from the admixture of the foregoing was useful as an injectable pharmaceutical composition.

### EXAMPLE “47.1”

Admixed were the following: THC 9.4 mg; Water for Injection 4.0 mL; Ethanol 0.2 mL; Tween 80 0.506 g; Corn oil 0.255 g. The composition resulting from the admixture of the foregoing was useful as an injectable pharmaceutical composition.

### EXAMPLE “47.11”

Admixed were the following: THC 10.4 mg; Water for Injection 4.3 mL; Ethanol 0.1 g; Tween 80 0.1 g; Corn oil 0.506 g. The composition resulting from the admixture of the foregoing was useful as an injectable pharmaceutical composition.

### EXAMPLE “47.12”

Admixed were the following: THC 6.6 mg; Ethanol 0.5 g; Propylene glycol 4.5 g. The composition resulting from the admixture of the foregoing was useful as an injectable pharmaceutical composition.

### EXAMPLE “47.13”

Admixed were the following: THC 6.9 mg; Water for Injection 1.0 g; Ethanol 0.500 g; Propylene glycol 3.5 mL. The composition resulting from the admixture of the foregoing was useful as an injectable pharmaceutical composition.

### EXAMPLE “47.14”

Admixed were the following: THC 5.8 mg; Water for Injection 2.4 mL; Ethanol 0.5 mL; Propylene glycol 2.0 mL; Tween 80 0.1 g; Hydroxypropyl beta-cyclodextrin 1.0 g. The composition resulting from the admixture of the foregoing was useful as an injectable pharmaceutical composition.

### EXMPLE “47.6”

Admixed were the following: THC 6.5 mg; Water for Injection 4.40 mL; Ethanol 0.200 mL; Propylene glycol 0.260 g; Tween 80 0.105 g. The composition resulting from the admixture of the foregoing was useful as an injectable pharmaceutical composition.

### EXAMPLE “47.7”

Admixed were the following: THC 6 mg; Water for Injection 4.59 mL; Ethanol 0.200 mL; PEG 400 0.260 g. The composition resulting from the admixture of the foregoing was useful as an injectable pharmaceutical composition.

### EXAMPLE “47.8”

Admixed were the following: THC 5 mg; Ethanol 0.200 mL; Pharmasolve 0.50 mL; Tween 80 0.106 mL; Water for Injection q.s. 5 mL. The composition resulting from the admixture of the foregoing was useful as an injectable pharmaceutical composition.

### EXAMPLE “47.9”

Admixed were the following: THC 6.0 mg; Ethanol 0.200 mL; Tween 80 0.106 g; Cremophor EL 10%; Water for Injection q.s. 5 mL. The composition resulting from the admixture of the foregoing was useful as an injectable pharmaceutical composition.

### EXAMPLE “48.9”

An aqueous composition was made by admixing THC with Ethanol, Cremophor EL, Tween 80 and Water for Injection such that the final concentration was 1.2 mg/mL THC; Ethanol 4%; Cremophor EL 10%; Tween 80 2%. The composition resulting from the admixture of the foregoing was useful as an injectable pharmaceutical composition.

### EXAMPLE “48.1”

An aqueous composition was made by admixing THC with Ethanol, Corn oil, Tween 80 and Water for Injection such that the final concentration was 1.88 mg/mL THC; Ethanol 4%; Corn oil 5%; Tween 80 10%. The composition resulting from the admixture of the foregoing was useful as an injectable pharmaceutical composition.

### EXAMPLE “48.11”

An aqueous composition was made by admixing THC wih Ethanol, Corn oil, Tween 80 and Water for Injection such that the final concentration was 2.08 mg/mL THC; Ethanol 2%; Corn oil 10%; Tween 80 2%. The composition resulting from the admixture of the foregoing was useful as an injectable pharmaceutical composition.

Example exemplifying method of making composition according to the invention. A preferred method of admixture was as follows: admixing tetrahydrocannabinol with ethanol to form a first mixture; admixing water with a pharmaceutically acceptable amphiphilic excipient to form a second mixture; and admixing the first mixture with the second mixture to form a third mixture. The third mixture was useful as an injectable pharmaceutical composition.

Example exemplifying method of using composition according to the invention. A preferred method of using a composition according to the invention is as follows: An subject presents with emesis, anorexia, or chronic or AIDS-related wasting syndrome. It is desire to treat, to lessen, or to ameliorate the emesis, anorexia, or chronic or AIDS-related wasting syndrome with which the subject presents. Accordingly, administered to the subject, by injection, is a composition according to the invention. In a preferred embodiment, a composition in which the THC concentration is not greater than 0.35%, and, in a particularly preferred embodiment, not greater than 0.1% to 0.2%, is administered to the subject by injection, whereupon the emesis, anorexia, or chronic or AIDS-related wasting syndrome is treated, lessened, or ameliorated in the subject.

Other examples and embodiments. The properties of the foregoing compositions are consistent with the notion that formulations including components at somewhat larger concentrations, due to the exigencies of mixing and scale-up, are within the scope of the invention. In such further embodiments and examples, in general, the concentration of tetrahydrocannabinol is, by mass, not greater than about 0.35%; the concentration of ethanol is, by mass, not greater than about 15%; the concentration of water is, by mass, not greater than about 90%; the concentration of Cremophor EL is, by mass, not greater than about 20%; the concentration of Polysorbate 80 is, by mass, not greater than about 15%; the concentration of Poloxamer 407 is, by mass, not greater than about 2.5%; the concentration of Poloxamer 237 is, by mass, not greater than about 5%; the concentration of PEG 400 is, by mass, not greater than about 20%; the concentration of Pharmasolve is, by volume, not greater than about 10%; the concentration of propylene glycol is, by mass, not greater than about 60%; the concentration of hyroxypropyl beta-cyclodextrin is, by mass, not greater than about 30%; the concentration of the salt renders the composition essentially isotonic; and the concentration of corn oil is, by mass, not greater than about 10%.

However, each of the foregoing embodiments is merely exemplary and is not intended to limit the scope of the invention, which encompasses all foreseeable and unforeseeable equivalents of what is described herein.

We claim:

1. An injectable pharmaceutical composition comprising tetrahydrocannabinol, ethanol, water, and a pharmaceutically acceptable amphiphilic excipient.

2. A composition according to claim 1 further comprising a pharmaceutically acceptable excipient salt.

3. A composition according to claim 1 further comprising a pharmaceutically acceptable excipient oil.

4. A composition according to claim 1 further comprising a pharmaceutically acceptable excipient antioxidant.

5. A composition according to claim 1, wherein the concentration of tetrahydrocannabinol is, by mass, not greater than about 0.35%.

6. A composition according to claim 1, wherein the concentration of ethanol is, by mass, not greater than about 15%.

7. A composition according to claim 1, wherein the concentration of water is, by mass, not greater than about 90%.

8. A composition according to claim 1, wherein the amphiphilic excipient comprises at least one member of the group consisting of: Cremophor EL, Polysorbate 80, Poloxamer 407, Poloxamer 237, PEG 400, Pharmasolve, propylene glycol, and hydroxypropyl beta-cyclodextrin.

9. A composition according to claim 2, wherein the salt comprises sodium chloride or sodium hydroxide.

10. A composition according to claim 3, wherein the oil comprises corn oil.

11. A composition according to claim 4, wherein the antioxidant comprises sodium metabisulfite or ascorbyl palmitate.

12. A composition according to claim 8, wherein the concentration of Cremophor EL is, by mass, not greater than about 20%.

13. A composition according to claim 8, wherein the concentration of Polysorbate 80 is, by mass, not greater than about 15%.

14. A composition according to claim 8, wherein the concentration of Poloxamer 407 is, by mass, not greater than about 2.5%.

15. A composition according to claim 8, wherein the concentration of Poloxamer 237 is, by mass, not greater than about 5%.

16. A composition according to claim 8, wherein the concentration of PEG 400 is, by mass, not greater than about 20%.

17. A composition according to claim 8, wherein the concentration of Pharmasolve is, by volume, not greater than about 10%.

18. A composition according to claim 8, wherein the concentration of propylene glycol is, by mass, not greater than about 60%.

19. A composition according to claim 8, wherein the concentration of hyroxypropyl beta-cyclodextrin is, by mass, not greater than about 30%.

20. A composition according to claim 9, wherein the concentration of the salt renders the composition essentially isotonic.

21. A composition according to claim 9, wherein the concentration of sodium chloride is, by mass, about 0.9%.

22. A composition according to claim 10, wherein the concentration of corn oil is, by mass, not greater than about 10%.

23. A method for manufacture of an injectable pharmaceutical composition comprising tetrahydrocannabinol, ethanol, water, and a pharmaceutically acceptable amphiphilic excipient, said method comprising the steps of: admixing tetrahydrocannabinol with ethanol to form a first mixture; admixing water with a pharmaceutically acceptable amphiphilic excipient to form a second mixture; and admixing the first mixture with the second mixture to form a third mixture, wherein said third mixture comprises an intermediate or a finished product in the manufacture of the injectable pharmaceutical composition.

24. A method of treating, lessening, or ameliorating emesis, anorexia, or chronic or AIDS-related wasting syndrome in a subject in which it is desired to treat, to lessen, or to ameliorate emesis, anorexia, or chronic or AIDS-related wasting syndrome, said method comprising administering to the subject a therapeutically effective amount of a composition according to claim 1.
